(19) United States
(12) Patent Application Publication
(10) Pub. No.: US 2011/0015724 A1
(43) Pub. Date: Jan. 20, 2011

(54) MEDICAL DEVICE HAVING HYDROPHILIC COATINGS

(75) Inventors: Jürgen Köcher, Langenfeld (DE); Thorsten Rische, Columbus, GA (US)

Correspondence Address: CONNOLLY BOVE LODGE & HUTZ, LLP, P O BOX 2207, WILMINGTON, DE, 19899, US

(73) Assignee: Bayer MaterialScience AG, Leverkusen (DE)

(21) Appl. No.: 12/933,299

(22) PCT Filed: Mar. 16, 2009
(86) PCT No.: PCT/EP09/01901

§ 371 Date: Sep. 17, 2010

(30) Foreign Application Priority Data
Mar. 20, 2008 (DE) 08153057.8

(51) Int. Cl. A61F 2/82; A61M 27/00; A61M 29/00; A61M 16/04; A61M 25/10; A61P 43/00; C09D 175/04; B05D 3/00

(52) U.S. Cl. 623/1.42; 604/544; 606/192; 128/207.14; 604/103.02; 524/590; 427/2.24; 424/423

(57) ABSTRACT

The present invention relates to a medical device having a coating comprising at least one polyurethane urea, wherein the coating comprises at least one polyurethane urea terminated with a copolymer unit of polyethyloxide and polypropyloxide.

The present invention relates to medical devices having hydrophilic and blood-compatible coatings comprising polyurethaneureas. These medical devices with enhanced surface qualities offer advantages in application by virtue of reduced friction and of their capacity, on contact with blood, to reduce the risk of blood clots.

The utilization of medical devices, such as of catheters, can be improved greatly through the equipping thereof with hydrophilic surfaces. The insertion and displacement of urinary or blood vessel catheters is made easier by the adsorption of a film of water by hydrophilic surfaces in contact with blood or urine. This reduces the friction between the catheter surface and the vessel walls, making the catheter easier to insert and move. Direct watering of the devices prior to the intervention can also be carried out, in order to reduce the friction through the formation of a homogeneous water film. The patients concerned have less pain, and the risk of injury to the vessel walls is reduced as a result. Furthermore, when catheters are used in contact with blood, there is always a risk of blood clots forming. In this context, hydrophilic coatings are considered generally to be useful for antithrombogenic coatings.

Catheters with hydrophilically treated surfaces are known per se from the prior art.

WO 99/38545 A1, for example, describes catheters which in a first embodiment are composed of a basecoating and a lubricious hydrophilic coating. Said prior art also describes, furthermore, an embodiment in which only a lubricious coating, i.e. a coating system without basecoating, is used. In that case a lubricious coating of a polyurethane is used. The isocyanate is utilized as a linking element on the surface for the attachment of hydrophilic groups. On the medical device, therefore, there are toxic isocyanates, and in order to accelerate curing it is necessary to employ highly toxic catalysts containing tin.

Known from WO 2006/037321 A1 are medical devices having a moistened hydrophilic surface which is intended to enhance the slip properties of the device. The surface is formed by a coating composition with a hydrophilic polymer and a moistening agent, comprising water and at least one lubricant. The coating composition known from this prior art is composed of a plurality of constituents, all of which must cooperate functionally in order to provide the resulting coating with the desired properties.

US 2003/0203991 A1 discloses hydrophilic coating materials which are based on mixtures of hydrophobic with hydrophilic polymers. Corresponding coating compositions for medical devices comprise (a) an aqueous polymeric matrix; (b) a hydrophilic polymer; (c) a colloidal metal oxide; and (d) a crosslinker. The requisite hydrophilicity of the coating according to US 2003/0203991 A1 is achieved by the polymer (b), which is incorporated into the corresponding polymeric matrix. Among the polymeric matrices used, but not used as a hydrophilic polymer, are polyurethane dispersions. The extensive ionic modification of these polyurethane dispersions can lead to an unwanted reduction in the hydrophilicity.

Mixtures of polyurethanes and polyvinylpyrrolidone as the hydrophilicizing constituent are described, furthermore, in U.S. Pat. No. 5,061,424. Moreover, U.S. Pat. No. 5,041,100 and US 2005/054774 A1 each describe polyurethane-containing coating compositions with polyethylene oxide (U.S. Pat. No. 5,041,100) or acrylates (US 2005/054774) as the hydrophilicizing constituents.

US 2006/040253 A1 describes hydrophilic coating of medical devices for the purpose of improving the slip properties, the composition comprising at least one water-soluble lubricious polymer and an insoluble polymer. The water-soluble lubricious polymer is selected inter alia from the group consisting of polyethylene oxide, polypropylene oxide, polyethyl vinyl alcohol, polyethyl vinyl acetate and polyvinylpyrrolidone, while the insoluble polymer is formed inter alia by polyurethanes, polyesterurethanes and polyetherurethanes.

Aliphatic polyetherpolyurethanes for hydrophilic coatings are likewise available commercially, an example being Tecogel® (Thermedics Polymer Products) or Hydroslip® (CardioTech International Inc.).

Not only the mixtures described in the literature but also the polyetherpolyurethanes available commercially have a variety of disadvantages. For instance, these mixtures are multi-component systems; in other words, they comprise two or more separate coatings, and they are therefore complicated to prepare, including more particularly those systems which are synthesized by covalent linking of two polymers (cf. US 2003/0203991 A1). The aliphatic polyetherurethanes are easier to use, but can often be processed only with fractions of organic solvents. This, however, is undesirable in the context of the application of medical devices more particularly in human or animal bodies, owing to the risk of the release of solvent residues from the coatings. Accordingly there is in principle still a need for medical devices for use within the human or animal body that have hydrophilic surfaces, and preferably eliminate the highlighted disadvantages of the prior art.

In this context, U.S. Pat. No. 5,589,563 recommends surface-modified end groups for biomedical polymers which can be used to coat medical devices. These polymers include different end groups, selected from amines, fluorinated alkanols, polydimethylsiloxanes and amine-terminated polyethylene oxides. As a coating for medical devices, however, these polymers lack satisfactory properties, particularly in respect of the required hydrophilicity.

It is an object of the present invention, therefore, to provide medical devices with hydrophilic surfaces. Since these surfaces are frequently used in blood contact, the surfaces of these materials ought also to possess good blood compatibility and ought more particularly to reduce the risk of blood clots being formed.

This invention provides medical devices with hydrophilic surfaces which can be produced by coating with specific polyurethane dispersions.

The medical devices of the invention comprise at least one coating comprising at least one polyurethaneurea which is terminated with a copolymer unit comprising polyethylene oxide and polypropylene oxide.

In accordance with the invention it has been found that compositions comprising these specific polyurethaneureas are outstandingly suitable as coatings on medical devices, to which they give an outstanding lubricous coating and at the same time reduce the risk of blood clots forming during treatment with the medical device.

Polyurethaneureas for the purposes of the present invention are polymeric compounds which have
(a) repeat units containing at least two urethane groups, of the following general structure

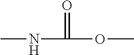

and
at least one repeat unit containing urea groups

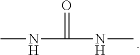

The coating compositions for use in accordance with the invention are based on polyurethaneureas which have substantially no ionic modification. By this is meant, in the context of the present invention, that the polyurethaneureas for use in accordance with the invention have essentially no ionic groups, such as, more particularly, no sulphonate, carboxylate, phosphate and phosphonate groups.

The term “essentially no ionic modification” means, in the context of the present invention, that any ionic modification is present at most in a fraction of 2.50% by weight, preferably at most 2.00% by weight, more particularly at most 1.50% by weight, more preferably at most 1.00% by weight, especially at most 0.50% by weight, the most preferred situation being for there to be no ionic modification at all of the polyurethaneurea provided in accordance with the invention.

The polyurethaneureas provided in accordance with the invention for the coating of the medical devices are preferably substantially linear molecules, but may also be branched, although this is less preferred. By substantially linear molecules are meant systems with a low level of incipient crosslinking, comprising a polycarbonate polyol having an average hydroxyl functionality of preferably 1.7 to 2.3, more particularly 1.8 to 2.2, more preferably 1.9 to 2.1. Systems of this kind can still be dispersed to a sufficient extent.

The number-average molecular weight of the polyurethaneureas used with preference in accordance with the invention is preferably 1000 to 200 000, more preferably from 5000 to 100 000. The number-average molecular weight here is measured against polystyrene as standard in dimethylacetamide at 30° C.

The average particle size of the dispersed polyurethaneureas of the invention is preferably 10 to 1000 nm, more preferably 20 to 800 nm, very preferably 50 to 600 nm.

### Polyurethaneureas

The polyurethaneurea-based coating systems for use in accordance with the invention are described in more detail below.

The polyurethaneureas used in accordance with the invention in the coatings of medical devices are prepared by reaction of synthesis components which encompass at least one polycarbonate polyol component, one polyisocyanate component, one polyoxyalkylene ether component, one diamine and/or amino alcohol component and, if desired, one polyol component.

The individual synthesis components are now described in more detail below.

### (a) Polycarbonate Polyol

The composition of the polyurethaneurea coating provided in accordance with the invention comprises units which originate from at least one hydroxyl-containing polycarbonate (polycarbonate polyol).

Suitable in principle for the introduction of units based on a hydroxyl-containing polycarbonate are polycarbonate polyols, i.e. polyhydroxy compounds, having an average hydroxyl functionality of 1.7 to 2.3, preferably of 1.8 to 2.2, more preferably of 1.9 to 2.1. The polycarbonate is therefore preferably of substantially linear construction and has only a slight three-dimensional crosslinking.

Suitable hydroxyl-containing polycarbonates are polycarbonates of a molecular weight (molecular weight determined via the OH number; DIN 53240) of preferably 400 to 6000 g/mol, more preferably 500 to 5000 g/mol, more particularly of 600 to 3000 g/mol, which are obtainable, for example, through reaction of carbonic acid derivatives, such as diphenyl carbonate, dimethyl carbonate or phosgene, with polyols, preferably diols. Examples of suitable such diols include ethylene glycol, 1,2- and 1,3-propanediol, 1,3- and 1,4-butanediol, 1,6-hexanediol, 1,8-octanediol, neopentyl glycol, 1,4-bishydroxymethylcyclohexane, 2-methyl-1,3-propanediol, 2,2,4-trimethylpentane-1,3-diol, di-, tri- or tetraethylene glycol, dipropylene glycol, polypropylene glycols, dibutylene glycol, polybutylene glycols, bisphenol A, tetrabromobisphenol A, and also lactone-modified diols.

The diol component preferably contains 40% to 100% by weight of hexanediol, preferably 1,6-hexanediol and/or hexanediol derivatives, preferably those which as well as terminal OH groups contain ether or ester groups, examples being products obtained by reaction of 1 mol of hexanediol with at least one 1 mol, preferably 1 to 2 mol, of caprolactone or through etherification of hexanediol with itself to give the di- or trihexylene glycol. Polyether-polycarbonate diols as well can be used. The hydroxyl polycarbonates ought to be substantially linear. If desired, however, they may be slightly branched as a result of the incorporation of polyfunctional components, more particularly low molecular weight polyols. Examples of those suitable for this purpose include glycerol, trimethylolpropane, hexane-1,2,6-triol, butane-1,2,4-triol, trimethylolpropane, pentaerythritol, quinitol, mannitol, sorbitol, methylglycoside or 1,3,4,6-dianhydrohexitols. Preferred polycarbonates are those based on hexane-1,6-diol, and also on co-diols with a modifying action such as butane-1,4-diol, for example, or else on ε-caprolactone. Further preferred polycarbonate diols are those based on mixtures of hexane-1,6-diol and butane-1,4-diol.

### (b) Polyisocyanate

The composition of the polyurethaneurea coating provided in accordance with the invention has units which originate from at least one polyisocyanate.

As polyisocyanates (b) it is possible to use all of the aromatic, araliphatic, aliphatic and cycloaliphatic isocyanates that are known to the skilled person and have an average NCO functionality ≧1, preferably ≧2, individually or in any desired mixtures with one another, irrespective of whether they have been prepared by phosgene or phosgene-free processes. They may also contain iminooxadiazinedione, isocyanurate, uretdione, urethane, allophanate, biuret, urea, oxadiazinetrione, oxazolidinone, acylurea and/or carbodiimide structures. The polyisocyanates may be used individually or in any desired mixtures with one another.

Preference is given to using isocyanates from the series of the aliphatic or cycloaliphatic representatives, which have a carbon backbone (without the NCO groups present) of 3 to 30, preferably 4 to 20, carbon atoms.

Particularly preferred compounds of component (b) conform to the type specified above having aliphatically and/or cycloaliphatically attached NCO groups, such as, for example, bis(isocyanatoalkyl)ethers, bis- and tris(isocyanatoalkyl)benzenes, -toluenes, and -xylenes, propane diisoscyanates, butane diisocyanates, pentane diisocyanates, hexane diisocyanates (e.g. hexamethylene diisocyanate, HDI), heptane diisocyanates, octane diisocyanates, nonane diisocyanates (e.g. trimethyl-HDI (TMDI), generally as a mixture of the 2,4,4 and 2,2,4 isomers), nonane triisocyanates (e.g. 4-isocyanatomethyl-1,8-octane diisocyanate), decane diisocyanates, decane triisocyanates, undecane diisocyanates, undecane triisocyanates, dodecane diisocyanates, dodecane triisocyanates, 1,3- and 1,4-bis(isocyanatomethyl)cyclohexanes (H₆XDI), 3-isocyanatomethyl-3,5,5-trimethylcyclohexyl isocyanate (isophorone diisocyanate, IPDI), bis(4-isocyanatocyclohexyl)methane (H₁₂MDI) or bis(isocyanatomethyl)norbornane (NBDI).

Very particularly preferred compounds of component (b) are hexamethylene diisocyanate (HDI), trimethyl-HDI (TMDI), 2-methylpentane 1,5-diisocyanate (MPDI), isophorone diisocyanate (IPDI), 1,3- and 1,4-bis(isocyanatomethyl)cyclohexane (H₆XDI), bis(isocyanato-methyl)norbornane (NBDI), 3(4)-isocyanatonnethyl-1-methyl-cyclohexyl isocyanate (IMCI) and/or 4,4′-bis(isocyanatocyclohexyl)methane (H₁₂MDI) or mixtures of these isocyanates. Further examples are derivatives of the above diisocyanates with a uretdione, isocyanurate, urethane, allophanate, biuret, iminooxadiazinedione and/or oxadiazinetrione structure and with more than two NCO groups.

The amount of constituent (b) in the coating composition for use in accordance with the invention is preferably 1.0 to 4.0 mol, more preferably 1.2 to 3.8 mol, more particularly 1.5 to 3.5 mol, based in each case on the constituent (a) of the coating composition for use in accordance with the invention.

### (c) Polyoxyalkylene Ethers

The polyurethaneurea used in the present invention has units which originate from a copolymer comprising polyethylene oxide and polypropylene oxide. These copolymer units are present in the form of end groups in the polyurethaneurea.

Nonionically hydrophilicizing compounds (c) are, for example, monofunctional polyalkylene oxide polyether alcohols containing an average 5 to 70, preferably 7 to 55, ethylene oxide units per molecule, of the kind available in conventional manner through alkoxylation of suitable starter molecules (e.g. in Ullmanns Enzyklopädie der technischen Chemie, 4th Edition, Volume 19, Verlag Chemie, Weinheim pp. 31-38).

Examples of suitable starter molecules are saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, such as diethylene glycol monobutyl ether, for example, unsaturated alcohols such as allyl alcohol, 1,1-dimethylallyl alcohol or oleyl alcohol, aromatic alcohols such as phenol, the isomeric cresols or methoxyphenols, araliphatic alcohols such as benzyl alcohol, anisyl alcohol or cinnamyl alcohol, secondary monoamines such as dimethylamine, diethylamine, dipropylamine, diisopropylamine, dibutylamine, bis(2-ethylhexyl)amine, N-methyl- and N-ethylcyclohexylamine or dicyclohexylamine, and also heterocyclic secondary amines such as morpholine, pyrrolidine, piperidine or 1H-pyrazole. Preferred starter molecules are saturated monoalcohols. Particular preference is given to using diethylene glycol monobutyl ether as a starter molecule.

The alkylene oxides, ethylene oxide and propylene oxide, can be used in any order or else in a mixture in the alkoxylation reaction.

The polyalkylene oxide polyether alcohols are mixed polyalkylene oxide polyethers of ethylene oxide and propylene oxide, whose alkylene oxide units are composed preferably to an extent of at least 30 mol %, more preferably at least 40 mol %, of ethylene oxide units. Preferred non-ionic compounds are monofunctional mixed polyalkylene oxide polyethers which contain at least 40 mol % of ethylene oxide units and not more than 60 mol % of propylene oxide units.

The average molar weight of the polyoxyalkylene ether is preferably 500 g/mol to 5000 g/mol, more preferably 1000 g/mol to 4000 g/mol, more preferably 1000 to 3000 g/mol.

The amount of constituent (c) in the coating composition for use in accordance with the invention is preferably 0.01 to 0.5 mol, more preferably 0.02 to 0.4 mol, more particularly 0.04 to 0.3 mol, based in each case on constituent (a) of the coating composition for use in accordance with the invention.

In accordance with the invention it has been possible to show that the polyurethaneureas with end groups based on mixed polyalkylene ethers comprising polyethylene oxide and polypropylene oxide are especially suitable for producing coatings having a high hydrophilicity. As will be shown later on below, in comparison to polyurethaneureas terminated only by polyethylene oxide, the coatings of the invention have the effect of a significantly low contact angle and are therefore more hydrophilic in form.

### (d) Diamine or Amino Alcohol

The composition of the polyurethaneurea coating provided in accordance with the invention includes units which originate from at least one diamine or amino alcohol.

The polyurethane coatings of the invention are produced using what are called chain extenders (d). Such chain extenders are diamines or polyamines and also hydrazides, e.g. hydrazine, 1,2-ethylenediamine, 1,2- and 1,3-diaminopropane, 1,4-diaminobutane, 1,6-diaminohexane, isophoronediamine, isomer mixture of 2,2,4- and 2,4,4-trimethylhexame-thylenediamine, 2-methylpentamethylenediamine, diethylenetriamine, 1,3- and 1,4-xylylene-diamine, α,α,α′,α′-tetramethyl-1,3- and -1,4-xylylenediamine and 4,4-diamino-dicyclohexylmethane, dimethylethylenediamine, hydrazine, adipic dihydrazide, 1,4-bis(aminomethyl)cyclohexane, 4,4′-diamino-3,3′-dimethyldicyclohexylmethane and other (C₁-C₄) di- and tetraalkyldicyclohexylmethanes, e.g. 4,4′-diamino-3,5-diethyl-3′,5′-diisopropyl-dicyclohexylmethane.

Suitable diamines or amino alcohols are generally low molecular weight diamines or amino alcohols which contain active hydrogen with differing reactivity towards NCO groups, such as compounds which as well as a primary amino group also contain secondary amino groups or which as well as an amino group (primary or secondary) also contain OH groups. Examples of such compounds are primary and secondary amines, such as 3-amino-1-methylaminopropane, 3-amino-1-ethylaminopropane, 3-amino-1-cyclohexylaminopropane, 3-amino-1-methylaminobutane, and also amino alcohols, such as N-aminoethylethanolamine, ethanolamine, 3-aminopropanol, neopentanolamine and, with particular preference, diethanolamine.

The constituent (d) of the coating composition for use in accordance with the invention can be used, in the context of the preparation of the composition, as a chain extender and/or as a form of chain termination.

The amount of constituent (d) in the coating composition for use in accordance with the invention is preferably 0.05 to 3.0 mol, more preferably 0.1 to 2.0 mol, more particularly 0.2 to 1.5 mol, based in each case on constituent (a) of the coating composition for use in accordance with the invention.

### (e) Polyols

In a further embodiment the composition of the polyurethaneurea coating of the invention comprises further units which originate from at least one further polyol.

The further low molecular weight polyols (e) used to synthesis the polyurethaneureas have the effect, generally, of stiffening and/or branching the polymer chain. The molecular weight is preferably 62 to 500 g/mol, more preferably 62 to 400 g/mol, more particularly 62 to 200 mol.

Suitable polyols may contain aliphatic, alicyclic or aromatic groups. Mention may be made here, for example, of the low molecular weight polyols having up to about 20 carbon atoms per molecule, such as ethylene glycol, diethylene glycol, triethylene glycol, 1,2-propanediol, 1,3-propanediol, 1,4-butanediol, 1,3-butylene glycol, cyclohexanediol, 1,4-cyclohexanedime-thanol, 1,6-hexanediol, neopentyl glycol, hydroquinone dihydroxyethyl ether, bisphenol A (2,2-bis(4-hydroxyphenyl)propane), hydrogenated bisphenol A (2,2-bis(4-hydroxy-cyclohexyl)propane), and also trimethylolpropane, glycerol or pentaerythritol, and mixtures of these and, if desired, other low molecular weight polyols as well. Use may also be made of ester diols such as, for example, α-hydroxybutyl-ε-hydroxy-caproic acid ester, ω-hydroxyhexyl-γ-hydroxybutyric acid ester, adipic acid (β-hydroxyethyl) ester or terephthalic acid bis(β-hydroxyethyl) ester.

The amount of constituent (e) in the coating composition for use in accordance with the invention is preferably 0.1 to 1.0 mol, more preferably 0.2 to 0.9 mol, more particularly 0.2 to 0.8 mol, based in each case on constituent (a) of the coating composition for use in accordance with the invention.

(f) Further Amine- and/or Hydroxy-Containing Units (Synthesis Component)

The reaction of the isocyanate-containing component (b) with the hydroxy- or amine-functional compounds (a), (c), (d) and, if used, (e) takes place typically with a slight NCO excess observed over the reactive hydroxy or amine compounds. As a result of dispersion in water, residues of isocyanate groups are hydrolysed to amine groups. In the specific case, however, it may be important to block the remaining residue of isocyanate groups before the polyurethane is dispersed.

The polyurethaneurea coatings provided in accordance with the invention may therefore also comprise synthesis components (f), which are located in each case at the chain ends and cap them. These units derive on the one hand from monofunctional compounds that are reactive with NCO groups, such as monoamines, more particularly mono-secondary amines, or monoalcohols.

Mention may be made here, for example, of ethanol, n-butanol, ethylene glycol monobutyl ether, 2-ethylhexanol, 1-octanol, 1-dodecanol, 1-hexadecanol, methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine and suitable substituted derivatives thereof.

Since the units (f) are used essentially in the coatings of the invention to destroy the NCO excess, the amount required is dependent essentially on the amount of the NCO excess, and cannot be specified generally.

Furthermore, the polyurethaneurea coatings provided in accordance with the invention may comprise further constituents typical for the intended purpose, such as additives and fillers. An example of such are active pharmacological substances, medicaments and additives which promote the release of active pharmacological substances (drug-eluting additives).

Active pharmacological substances and medicaments which may be used in the coatings of the invention on the medical devices are in general, for example, thromboresistant agents, antibiotic agents, antitumour agents, growth hormones, antiviral agents, antiangiogenic agents, angiogenic agents, antimitotic agents, anti-inflammatory agents, cell cycle regulators, genetic agents, hormones, and also their homologues, derivatives, fragments, pharmaceutical salts, and combinations thereof.

Specific examples of such medicaments and active pharmacological substances hence include thromboresistant (non-thrombogenic) agents and other agents for suppressing acute thrombosis, stenosis or late restenosis of the arteries, examples being heparin, streptokinase, urokinase, tissue plasminogen activator, anti-thromboxan-B₂ agent; anti-B-thromboglobulin, prostaglandin-E, aspirin, dipyridimol, anti-thromboxan-A₂ agent, murine monoclonal antibody 7E3, triazolopyrimidine, ciprostene, hirudin, ticlopidine, nicorandil, etc. A growth factor can likewise be utilized as a medicament in order to suppress subintimal fibromuscular hyperplasia at the arterial stenosis site, or any other cell growth inhibitor can be utilized at the stenosis site.

The medicament or active pharmacological substance may also be composed of a vasodilatator, in order to counteract vasospasm—for example, an antispasm agent such as papaverine. The medicament may be a vaso active agent per se, such as calcium antagonists, or α- and β-adrenergic agonists or antagonists. In addition the therapeutic agent may be a biological adhesive such as cyanoacrylate in medical grade, or fibrin, which is used, for example, for bonding a tissue valve to the wall of a coronary artery.

The therapeutic agent may further be an antineoplastic agent such as 5-fluorouracil, preferably with a controlling releasing vehicle for the agent (for example, for the use of an ongoing controlled releasing antineoplastic agent at a tumour site).

The therapeutic agent may be an antibiotic, preferably in combination with a controlling releasing vehicle for ongoing release from the coating of a medical device at a localized focus of infection within the body. Similarly, the therapeutic agent may comprise steroids for the purpose of suppressing inflammation in localized tissue, or for other reasons.

Specific examples of suitable medicaments include:
(a) heparin, heparin sulphate, hirudin, hyaluroic acid, chondroitin sulphate, dermatan sulphate, keratin sulphate, lytic agents, including urokinase and streptokinase, their homologues, analogues, fragments, derivatives and pharmaceutical salts thereof;
(b) antibiotic agents such as penicillins, cephalosporins, vacomycins, aminoglycosides, quinolones, polymyxins, erythromycins; tetracyclines, chloramphenicols, clindamycins, lincomycins, sulphonamides, their homologues, analogues, derivatives, pharmaceutical salts and mixtures thereof;
(c) paclitaxel, docetaxel, immunosuppressants such as sirolimus or everolimus, alkylating agents, including mechlorethamine, chlorambucil, cyclophosphamide, melphalane and ifosfamide; antimetabolites, including methotrexate, 6-mercaptopurine, 5-fluorouracil and cytarabine; plant alkoids, including vinblastin; vincristin and etoposide; antibiotics, including doxorubicin, daunomycin, bleomycin and mitomycin; nitrosurea, including carmustine and lomustine; inorganic ions, including cisplatin; biological reaction modifiers, including interferon; angiostatins and endostatins; enzymes, including asparaginase; and hormones, including tamoxifen and flutamide, their homologues, analogues, fragments, derivatives, pharmaceutical salts and mixtures thereof; and
(d) antiviral agents such as amantadine, rimantadine, rabavirin, idoxuridine, vidarabin, trifluridine, acyclovir, ganciclovir, zidovudine, phosphonoformates, interferons, their homologues, analogues, fragments, derivatives, pharmaceutical salts and mixtures thereof; and
e) antiflammatory agents such as, for example, ibuprofen, dexamethasone or methylprednisolone.

In one preferred embodiment the coating composition provided in accordance with the invention comprises a polyurethaneurea which is synthesized from
a) at least one polycarbonate polyol;
b) at least one polyisocyanate;
c) at least one monofunctional mixed polyalkylene ether comprising polyethylene oxide and polypropylene oxide; and
d) at least one diamine or amino alcohol.

In a further preferred embodiment the coating composition of the invention comprises a polyurethaneurea which is synthesized from
a) at least one polycarbonate polyol;
b) at least one polyisocyanate;
c) at least one monofunctional mixed polyalkylene ether comprising polyethylene oxide and polypropylene oxide;
d) at least one diamine or amino alcohol; and
e) at least one polyol.

In a further embodiment of the present invention the coating composition provided in accordance with the invention comprises a polyurethaneurea which is synthesized from
a) at least one polycarbonate polyol;
b) at least one polyisocyanate;
c) at least one monofunctional mixed polyalkylene ether comprising polyethylene oxide and polypropylene oxide;
d) at least one diamine or amino alcohol;
e) at least one polyol; and
f) at least one amine- or hydroxyl-containing monomer which is located at the polymer chain ends.

Particular preference is given in accordance with the invention to coating the medical devices using polyurethaneureas which are synthesized from
a) at least one polycarbonate polyol having an average molar weight between 400 g/mol and 6000 g/mol and a hydroxyl functionality of 1.7 to 2.3, or mixtures of such polycarbonate polyols;
b) at least one aliphatic, cycloaliphatic or aromatic polyisocyanate or mixtures of such polyisocyanates in an amount per mole of the polycarbonate polyol of 1.0 to 4.0 mol;
c) at least one monofunctional mixed polyoxyalkylene ether comprising polyethylene oxide and polypropylene oxide or a mixture of such polyethers, having an average molar weight between 500 g/mol and 5000 g/mol, in an amount per mole of the polycarbonate polyol of 0.01 to 0.5 mol;
d) at least one aliphatic or cycloaliphatic diamine or at least one amino alcohol, as so-called chain extenders, or mixtures of such compounds in an amount per mole of the polycarbonate polyol of 0.05 to 3.0 mol;
e) if desired, one or more short-chain aliphatic polyols having a molar weight between 62 g/mol and 500 g/mol, in an amount per mole of the polycarbonate polyol of 0.1 to 1.0 mol; and
f) if desired, amine- or OH-containing units which are located on, and cap, the polymer chain ends.

Preference is further given in accordance with the invention to coating medical devices using polyurethaneureas which are synthesized from
a) at least one polycarbonate polyol having an average molar weight between 500 g/mol and 5000 g/mol and a hydroxyl functionality of 1.8 to 2.2, or of mixtures of such polycarbonate polyols;
b) at least one aliphatic, cycloaliphatic or aromatic polyisocyanate or mixtures of such polyisocyanates in an amount per mole of the polycarbonate polyol of 1.2 to 3.8 mol;
c) at least one monofunctional mixed polyoxyalkylene ether comprising polyethylene oxide and polypropylene oxide or a mixture of such polyethers, having an average molar weight between 1000 g/mol and 4000 g/mol, in an amount per mole of the polycarbonate polyol of 0.02 to 0.4 mol;
d) at least one aliphatic or cycloaliphatic diamine or at least one amino alcohol, as so-called chain extenders, or mixtures of such compounds in an amount per mole of the polycarbonate polyol of 0.1 to 2.0 mol;
e) if desired, one or more short-chain aliphatic polyols having a molar weight between 62 g/mol and 400 g/mol, in an amount per mole of the polycarbonate polyol of 0.2 to 0.9 mol; and
f) if desired, amine- or OH-containing units which are located on, and cap, the polymer chain ends.

Preference is also further given in accordance with the invention to coating catheter materials using polyurethaneureas which are synthesized from
a) at least one polycarbonate polyol having an average molar weight between 600 g/mol and 3000 g/mol and a hydroxyl functionality of 1.9 to 2.1, or of mixtures of such polycarbonate polyols;
b) at least one aliphatic, cycloaliphatic or aromatic polyisocyanate or mixtures of such polyisocyanates in an amount per mole of the polycarbonate polyol of 1.5 to 3.5 mol;
c) at least one monofunctional mixed polyoxyalkylene ether comprising polyethylene oxide and polypropylene oxide or a mixture of such polyethers, having an average molar weight between 1000 g/mol and 3000 g/mol, in an amount per mole of the polycarbonate polyol of 0.04 to 0.3 mol;
d) at least one aliphatic or cycloaliphatic diamine or at least one amino alcohol, as so-called chain extenders, or mixtures of such compounds in an amount per mole of the polycarbonate polyol of 0.2 to 1.5 mol;
e) if desired, one or more short-chain aliphatic polyols having a molar weight between 62 g/mol and 200 g/mol, in an amount per mole of the polycarbonate polyol of 0.2 to 0.8 mol; and
f) if desired, amine- or OH-containing units which are located on, and cap, the polymer chain ends.

The coating composition is applied to a medical device.

### Medical Device

The term “medical device” is to be understood broadly in the context of the present invention. Suitable, non-limiting examples of medical devices (including instruments) are contact lenses; cannulas; catheters, for example urological catheters such as urinary catheters or ureteral catheters; central venous catheters; venous catheters or inlet or outlet catheters; dilation balloons; catheters for angioplasty and biopsy; catheters used for introducing a stent, an embolism filter or a vena caval filter; balloon catheters or other expandable medical devices; endoscopes; laryngoscopes; tracheal devices such as endotracheal tubes, respirators and other tracheal aspiration devices; bronchoalveolar lavage catheters; catheters used in coronary angioplasty; guide rods, insertion guides and the like; vascular plugs; pacemaker components; cochlear implants; dental implant tubes for feeding, drainage tubes; and guide wires;

The coating solutions of the invention may be used, furthermore, for producing protective coatings, for example for gloves, stents and other implants; external (extracorporeal) blood lines (blood-carrying pipes); membranes; for example for dialysis; blood filters; devices for circulatory support; dressing material for wound management; urine bags and stoma bags. Also included are implants which comprise a medically active agent, such as medically active agents for stents or for balloon surfaces or for contraceptives.

Typically the medical device is formed from catheters, endoscopes, laryngoscopes, endotracheal tubes, feeding tubes, guide rods, stents, and other implants.

There are many materials suitable as a substrate of the surface to be coated, such as metals, textiles, ceramics or plastics, the use of plastics being preferred for the production of medical devices.

In accordance with the invention it has been found that it is possible to produce medical devices having very hydrophilic and hence lubricious, blood-compatible surfaces by using aqueous, nonionically stabilized polyurethane dispersions of the type described above to coat the medical devices. The coating compositions described above are obtained preferably as aqueous dispersions and are applied to the surface of the medical devices.

### Preparation of the Coating Solution

The constituents of the coatings, described in more detail above, are generally reacted such that first of all an isocyanate-functional prepolymer free of urea groups is prepared by reaction of the constituents (a), (b), (c) and, if desired, (e), the amount-of-substance ratio of isocyanate groups to isocyanate-reactive groups of the polycarbonate polyol being preferably 0.8 to 4.0, more preferably 0.9 to 3.8, more particularly 1.0 to 3.5.

In an alternative embodiment it is also possible first to react the constituent (a) separately with the isocyanate (b). Then, after that, constituents (c) and, if desired, (e) can be added and reacted. Subsequently, in general, the remaining isocyanate groups are given an amino-functional chain extension or termination, before, during or after dispersion in water, the ratio of equivalents of isocyanate-reactive groups of the compounds used for chain extension to free isocyanate groups of the prepolymer being preferably between 40% to 150%, more preferably between 50% to 120%, more particularly between 60% to 120% (constituent (d)).

The polyurethane dispersions of the invention are prepared preferably by the process known as the acetone process. For the preparation of the polyurethane dispersion by this acetone process, some or all of the constituents (a), (c) and (e), which must not contain any primary or secondary amino groups, and the polyisocyanate component (b) are typically introduced, for the preparation of an isocyanate-functional polyurethane prepolymer, and where appropriate are diluted with a water-miscible solvent which is nevertheless inert towards isocyanate groups, and the batch is heated to temperatures in the range from 50 to 120° C. To accelerate the isocyanate addition reaction it is possible to use the catalysts known in polyurethane chemistry, an example being dibutyltin dilaurate. Preference is given to synthesis without catalyst.

Suitable solvents are the typical aliphatic, keto-functional solvents such as, for example, acetone, butanone, which can be added not only at the beginning of the preparation but also, if desired, in portions later on as well. Acetone and butanone are preferred. Other solvents such as xylene, toluene, cyclohexane, butyl acetate, methoxypropyl acetate and solvents with ether units or ester units, for example, may likewise be used and may be removed in whole or in part by distillation or may remain entirely in the dispersion.

Subsequently any constituents of (c) and (e) not added at the beginning of the reaction are metered in.

In a preferred way, the prepolymer is prepared without addition of solvent and only for its chain extension is diluted with a suitable solvent, preferably acetone.

In the preparation of the polyurethane prepolymer, the amount-of-substance ratio of isocyanate groups to isocyanate-reactive groups is preferably 0.8 to 4.0, more preferably 0.9 to 3.8, more particularly 1.0 to 3.5.

The reaction to give the prepolymer takes place partially or completely, but preferably completely. In this way, polyurethane prepolymers which contain free isocyanate groups are obtained, in bulk or in solution.

Subsequently, in a further process step, if it has not yet taken place or has taken place only partly, the resulting prepolymer is dissolved by means of aliphatic ketones such as acetone or butanone.

Subsequently, possible NH₂—, NH-functional and/or OH-functional components are reacted with the remaining isocyanate groups. This chain extension/termination may be carried out alternatively in solvent prior to dispersing, during dispersing, or in water after dispersion has taken place. Preference is given to carrying out the chain extension prior to dispersing in water.

Where compounds conforming to the definition of (d) with NH₂ or NH groups are used for chain extension, the chain extension of the prepolymers takes place preferably prior to the dispersing.

The degree of chain extension, in other words the ratio of equivalents of NCO-reactive groups of the compounds used for chain extension to free NCO groups of the prepolymer, is preferably between 40% to 150%, more preferably between 50% to 120%, more particularly between 60% to 120%.

The aminic components (d) may if desired be used in water-diluted or solvent-diluted form in the process of the invention, individually or in mixtures, in which case any sequence of addition is possible in principle.

If water or organic solvents are used as diluents, the diluent content is preferably 70% to 95% by weight.

The preparation of the polyurethane dispersion from the prepolymers takes place following the chain extension. For this purpose, either the dissolved and chain-extended polyurethane polymer is introduced into the dispersing water, where appropriate with strong shearing, such as vigorous stirring, for example, or, conversely, the dispersing water is stirred into the prepolymer solutions. Preferably the water is added to the dissolved prepolymer.

The solvent still present in the dispersions after the dispersing step is typically then removed by distillation. Its removal during the actual dispersing is likewise a possibility.

The solids content of the polyurethane dispersion after the synthesis is between 20% to 70% by weight, preferably 20% to 65% by weight. For coating experiments these dispersions can be diluted arbitrarily with water, in order to allow the thickness of the coating to be varied. All concentrations from 1% to 60% by weight are possible; preference is given to concentrations in the 1% to 40% by weight range.

In this context it is possible to attain any desired coat thicknesses, such as, for example, from a few 100 nm up to a few 100 μm, although higher and lower thicknesses are possible in the context of the present invention.

The polyurethane materials for the coating of the medical devices can be diluted to any desired value by dilution of the aqueous dispersions of the invention with water. Furthermore, it is possible to add thickeners, in order, where appropriate, to allow the viscosity of the polyurethane dispersions to be increased. Further additions, such as antioxidants, buffer materials for adjusting the pH, or pigments, for example, are likewise possible. It is also possible if desired, furthermore, to use further additions such as hand assistants, dyes, matting agents, UV stabilizers, light stabilizers, hydrophobing agents, hydrophilicizing agents and/or flow control assistants.

### Production of the Coatings

In the context of the present invention it is more particularly preferred for the coatings of the medical devices to be produced starting from dispersions of the coating composition described in more detail above. The dispersion is preferably obtained as described above.

In accordance with the invention it has emerged that the resulting coatings on medical devices differ according to whether the coating is produced starting from a dispersion or from a solution.

The coatings of the invention on medical devices have advantages when they are obtained starting from dispersions of the above-described coating compositions, since dispersions of the coating systems of the invention lead to coatings on the medical devices that do not contain organic solvent residues, and therefore are generally unobjectionable from a toxicity standpoint, and at the same time lead to a more pronounced hydrophilicity, which is evident, for example, from a small contact angle. Reference is made on this point to the experiments, and comparative experiments, that are elucidated later on below.

In a further embodiment the present invention therefore provides a medical device having at least one hydrophilic coating comprising at least one polyurethaneurea, the coating being produced starting from a dispersion of the polyurethaneurea. The polyurethaneurea is preferably the above-described polyurethaneurea of the invention.

The medical devices of the invention can be coated with the hydrophilic polyurethane dispersions by means of a variety of methods. Examples of suitable coating techniques for this purpose include knifecoating, printing, transfer coating, spraying, spin coating or dipping.

The aqueous polyurethane dispersions which are used as starting material for producing the coatings can be prepared by any desired processes, although the above-described acetone process is preferred.

A wide variety of substrates can be coated in this context, such as metals, textiles, ceramics and plastics. Preference is given to coating medical devices manufactured from metals or from plastic. Examples of metals include the following: medical stainless steel or nickel titanium alloys. Many polymer materials are conceivable from which the medical device may be constructed, examples being polyamide; polystyrene; polycarbonate; polyethers; polyesters; polyvinyl acetate; natural and synthetic rubbers; block copolymers of styrene and unsaturated compounds such as ethylene, butylene and isoprene; polyethylene or copolymers of polyethylene and polypropylene; silicone; polyvinyl chloride (PVC) and polyurethanes. For better adhesion of the hydrophilic polyurethanes to the medical device, further suitable coatings may be applied as a base before these hydrophilic coating materials are applied.

In addition to the hydrophilic properties of the improvement of slip, the coating compositions provided in accordance with the invention are also distinguished by a high level of blood compatibility. As a result, working with these coatings is also advantageous, particularly in blood contact. In comparison to polymers of the prior art, the materials exhibit reduced coagulation tendency in blood contact.

The advantages of the catheters of the invention with the hydrophilic polyurethane coatings are set out by means of comparative experiments in the following examples.

### EXAMPLES

The NCO content of the resins described in the inventive and comparative examples was determined by titration in accordance with DIN EN ISO 11909.

The solids contents were determined in accordance with DIN-EN ISO 3251.1 g of polyurethane dispersion was dried at 115° C. to constant weight (15-20 min) using an infrared drier.

The average particle sizes of the polyurethane dispersions are measured using the High Performance Particle Sizer (HPPS 3.3) from Malvern Instruments.

Unless noted otherwise, amounts indicated in % are % by weight and relate to the aqueous dispersion obtained.

### Substances and Abbreviations Used:

Desmophen C2200: Polycarbonate polyol, OH number 56 mg KOH/g, number-average molecular weight 2000 g/mol (Bayer, MaterialScience AG, Leverkusen, Del.)
Desmophen C1200: Polycarbonate polyol, OH number 56 mg KOH/g, number-average molecular weight 2000 g/mol (Bayer MaterialScience AG, Leverkusen, Del.)
Desmophen XP 2613 Polycarbonate polyol, OH number 56 mg KOH/g, number-average molecular weight 2000 g/mol (Bayer MaterialScience AG, Leverkusen, Del.)
PolyTHF® 2000: Polytetramethylene glycol polyol, OH number 56 mg KOH/g, number-average molecular weight 2000 g/mol (BASF AG, Ludwigshafen, Del.)
Polyether LB 25: (monofunctional polyether based on ethylene oxide/propylene oxide, number-average molecular weight 2250 g/mol, OH number 25 mg KOH/g (Bayer MaterialScience AG, Leverkusen, Del.)

### Example 1

This example describes the preparation of an inventive polyurethaneurea dispersion.

277.2 g of Desmophen C 2200, 33.1 g of Polyether LB 25 and 6.7 g of neopentyl glycol were introduced at 65° C. and homogenized by stirring for 5 minutes. At 65° C., this mixture was admixed over the course of 1 minute first with 71.3 g of 4,4′-bis(isocyanato-cyclohexyl)methane (H₁₂MDI) and then with 11.9 g of isophorone diisocyanate. This mixture was heated to 110° C. After 3 h 40 min the theoretical NCO value was reached. The completed prepolymer was dissolved at 50° C. in 711 g of acetone and then at 40° C. a solution of 4.8 g of ethylene diamine in 16 g of water was metered in over the course of 10 min. The subsequent stirring time was 15 min. Subsequently, over the course of 15 min, a dispersion was carried out by addition of 590 g of water. After that the solvent was removed by distillation under reduced pressure. This gave a storage-stable polyurethane dispersion having a solids content of 41.5% and an average particle size of 164 nm.

### Example 2

This example describes the preparation of an inventive polyurethaneurea dispersion.

269.8 g of Desmophen C 2200, 49.7 g of Polyether LB 25 and 6.7 g of neopentyl glycol were introduced at 65° C. and homogenized by stirring for 5 minutes. At 65° C., this mixture was admixed over the course of 1 minute first with 71.3 g of 4,4′-bis(isocyanato-cyclohexyl)methane (H₁₂MDI) and then with 11.9 g of isophorone diisocyanate. This mixture was heated to 100° C. After 21.5 h the theoretical NCO value was reached. The completed prepolymer was dissolved at 50° C. in 711 g of acetone and then at 40° C. a solution of 4.8 g of ethylene diamine in 16 g of water was metered in over the course of 10 min. The subsequent stirring time was 5 min. Subsequently, over the course of 15 min, a dispersion was carried out by addition of 590 g of water. After that the solvent was removed by distillation under reduced pressure. This gave a storage-stable polyurethane dispersion having a solids content of 41.3% and an average particle size of 109 nm.

### Example 3

This example describes the preparation of an inventive polyurethaneurea dispersion.

277.2 g of Desmophen C1200, 33.1 g of Polyether LB 25 and 6.7 g of neopentyl glycol were introduced at 65° C. and homogenized by stirring for 5 minutes. At 65° C., this mixture was admixed over the course of 1 minute first with 71.3 g of 4,4′-bis(isocyanato-cyclohexyl)methane (H₁₂MDI) and then with 11.9 g of isophorone diisocyanate. This mixture was heated to 110° C. After 2.5 h the theoretical NCO value was reached. The completed prepolymer was dissolved at 50° C. in 711 g of acetone and then at 40° C. a solution of 4.8 g of ethylene diamine in 16 g of water was metered in over the course of 10 min. The subsequent stirring time was 5 min. Subsequently, over the course of 15 min, a dispersion was carried out by addition of 590 g of water. After that the solvent was removed by distillation under reduced pressure. This gave a storage-stable polyurethane dispersion having a solids content of 40.4% and an average particle size of 146 nm.

### Example 4

This example describes the preparation of an inventive polyurethaneurea dispersion.

282.1 g of Desmophen C 2200, 22.0 g of Polyether LB 25 and 6.7 g of neopentyl glycol were introduced at 65° C. and homogenized by stirring for 5 minutes. At 65° C., this mixture was admixed over the course of 1 minute first with 71.3 g of 4,4′-bis(isocyanato-cyclohexyl)methane (H₁₂MDI) and then with 11.9 g of isophorone diisocyanate. This mixture was heated to 110° C. After 21.5 h the theoretical NCO value was reached. The completed prepolymer was dissolved at 50° C. in 711 g of acetone and then at 40° C. a solution of 4.8 g of ethylene diamine in 16 g of water was metered in over the course of 10 min. The subsequent stirring time was 5 min. Subsequently, over the course of 15 min, a dispersion was carried out by addition of 590 g of water. After that the solvent was removed by distillation under reduced pressure. This gave a storage-stable polyurethane dispersion having a solids content of 41.7% and an average particle size of 207 nm.

### Example 5

This example describes the preparation of an inventive polyurethaneurea dispersion.

269.8 g of Desmophen XP 2613, 49.7 g of Polyether LB 25 and 6.7 g of neopentyl glycol were introduced at 65° C. and homogenized by stirring for 5 minutes. At 65° C., this mixture was admixed over the course of 1 minute first with 71.3 g of 4,4′-bis(isocyanato-cyclohexyl)methane (H₁₂MDI) and then with 11.9 g of isophorone diisocyanate. This mixture was heated to 110° C. After 70 min the theoretical NCO value was reached. The completed prepolymer was dissolved at 50° C. in 711 g of acetone and then at 40° C. a solution of 4.8 g of ethylene diamine in 16 g of water was metered in over the course of 10 min. The subsequent stirring time was 5 min. Subsequently, over the course of 15 min, a dispersion was carried out by addition of 590 g of water. After that the solvent was removed by distillation under reduced pressure. This gave a storage-stable polyurethane dispersion having a solids content of 41.2% and an average particle size of 112 nm:

### Example 6

This example describes the preparation of an inventive polyurethaneurea dispersion.

249.4 g of Desmophen C 2200, 33.1 g of Polyether LB 25, 1.9 g of trimethylolpropane and 6.7 g of neopentyl glycol were introduced at 65° C. and homogenized by stirring for 5 minutes. At 65° C., this mixture was admixed over the course of 1 minute first with 71.3 g of 4,4′-bis(isocyanatocyclohexyl)methane (H₁₂MDI) and then with 11.9 g of isophorone diisocyanate. This mixture was heated to 110° C. After 4 h 20 min the theoretical NCO value was reached. The completed prepolymer was dissolved at 50° C. in 720 g of acetone and then at 40° C. a solution of 3.3 g of ethylene diamine in 16 g of water was metered in over the course of 10 min. The subsequent stirring time was 15 min. Subsequently, over the course of 15 min, a dispersion was carried out by addition of 590 g of water. After that the solvent was removed by distillation under reduced pressure. This gave a storage-stable polyurethane dispersion having a solids content of 38.9% and an average particle size of 144 nm.

### Example 7

282.1 g of Desmophen XP 2613, 22.0 g of Polyether LB 25 and 6.7 g of neopentyl glycol were introduced at 65° C. and homogenized by stirring for 5 minutes. At 65° C., this mixture was admixed over the course of 1 minute first with 71.3 g of 4,4′-bis(isocyanato-cyclohexyl)methane (H₁₂MDI) and then with 11.9 g of isophorone diisocyanate. This mixture was heated to 110° C. After 70 min the theoretical NCO value was reached. The completed prepolymer was dissolved at 50° C. in 711 g of acetone and then at 40° C. a solution of 4.8 g of ethylene diamine in 16 g of water was metered in over the course of 10 min. The subsequent stirring time was 5 min. Subsequently, over the course of 15 min, a dispersion was carried out by addition of 590 g of water. After that the solvent was removed by distillation under reduced pressure. This gave a storage-stable polyurethane dispersion having a solids content of 38.3% and an average particle size of 215 nm.

### Example 8

This example describes the preparation of a polyurethaneurea dispersion as a comparison product to the inventive Example 1. The Desmophen C2200 is replaced by PolyTHF 2000.

277.2 g of PolyTHF 2000, 33.1 g of Polyether LB 25 and 6.7 g of neopentyl glycol were introduced at 65° C. and homogenized by stirring for 5 minutes. At 65° C., this mixture was admixed over the course of 1 minute first with 71.3 g of 4,4′-bis(isocyanato-cyclohexyl)methane (H₁₂MDI) and then with 11.9 g of isophorone diisocyanate. This mixture was heated to 110° C. After 18 h the theoretical NCO value was reached. The completed prepolymer was dissolved at 50° C. in 711 g of acetone and then at 40° C. a solution of 4.8 g of ethylene diamine in 16 g of water was metered in over the course of 10 min. The subsequent stirring time was 5 min. Subsequently, over the course of 15 min, a dispersion was carried out by addition of 590 g of water. After that the solvent was removed by distillation under reduced pressure. This gave a storage-stable polyurethane dispersion having a solids content of 40.7% and an average particle size of 166 nm.

### Example 9

This example describes the preparation of a polyurethaneurea dispersion as a comparison product to the inventive Example 2. The Desmophen C2200 is replaced by the PolyTHF 2000.

269.8 g of PolyTHF 2000, 49.7 g of Polyether LB 25 and 6.7 g of neopentyl glycol were introduced at 65° C. and homogenized by stirring for 5 minutes. At 65° C., this mixture was admixed over the course of 1 minute first with 71.3 g of 4,4′-bis(isocyanato-cyclohexyl)methane (H₁₂MDI) and then with 11.9 g of isophorone diisocyanate. This mixture was heated to 100° C. After 17.5 h the theoretical NCO value was reached. The completed prepolymer was dissolved at 50° C. in 711 g of acetone and then at 40° C. a solution of 4.8 g of ethylene diamine in 16 g of water was metered in over the course of 10 min. The subsequent stirring time was 5 min. Subsequently, over the course of 15 min, a dispersion was carried out by addition of 590 g of water. After that the solvent was removed by distillation under reduced pressure. This gave a storage-stable polyurethane dispersion having a solids content of 41.6% and an average particle size of 107 nm.

### Example 10

This example describes the preparation of a polyurethaneurea dispersion as a comparison product to the inventive Example 4. The Desmophen C2200 is replaced by the PolyTHF 2000.

282.1 g of PolyTHF 2000, 22.0 g of Polyether LB 25 and 6.7 g of neopentyl glycol were introduced at 65° C. and homogenized by stirring for 5 minutes. At 65° C., this mixture was admixed over the course of 1 minute first with 71.3 g of 4,4′-bis(isocyanato-cyclohexyl)methane (H₁₂MDI) and then with 11.9 g of isophorone diisocyanate. This mixture was heated to 110° C. After 21.5 h the theoretical NCO value was reached. The completed prepolymer was dissolved at 50° C. in 711 g of acetone and then at 40° C. a solution of 4.8 g of ethylene diamine in 16 g of water was metered in over the course of 10 min. The subsequent stirring time was 5 min. Subsequently, over the course of 15 min, a dispersion was carried out by addition of 590 g of water. After that the solvent was removed by distillation under reduced pressure. This gave a storage-stable polyurethane dispersion having a solids content of 37.5% and an average particle size of 195 nm.

### Example 11

### Production of the Coatings and Measurement of the Static Contact Angle

The coatings for the measurement of the static contact angle were produced on glass slides measuring 25×75 mm using a spincoater (RC5 Gyrset 5, Karl Sass, Garching, Germany). For this purpose a slide was clamped onto the sample plate of the spincoater and covered homogeneously with about 2.5-3 g of aqueous undiluted polyurethane dispersion. Rotation of the sample plate at 1300 revolutions per minute for 20 sec gave a homogeneous coating, which was dried at 100° C. for 15 min and then at 50° C. for 24 h. The coated slides obtained were subjected directly to a contact angle measurement.

A static contact angle measurement is performed on the resulting coatings on the slides. Using the video contact angle measuring instrument OCA20 from Dataphysics, with computer-controlled injection, 10 drops of Millipore water are placed on the specimen, and their static wetting angle is measured. Beforehand, using an antistatic drier, the static charge (if present) on the sample surface is removed.

TABLE 1
Statistic contact angle measurements

| PU FILM | CONTACT ANGLE [°] |
|---|---|
| Inventive Example 1 | <10 |
| Inventive Example 2 | 11 |
| Inventive Example 3 | 14 |
| Inventive Example 4 | 20 |
| Inventive Example 5 | 14 |
| Inventive Example 6 | 26 |
| Inventive Example 7 | 41 |
| Comparative Example 8 | 66 |
| Comparative Example 9 | 62 |
| Comparative Example 10 | 77 |

As Table 1 shows, the polycarbonate-containing coatings of Inventive Examples 1 to 7 give extremely hydrophilic coatings with static contact angles≦45°. The coatings of Examples 1 to 6 produce extraordinarily hydrophilic coatings with static contact angles<30°. In contrast, the PolyTHF-containing coatings from Comparative Examples 7 to 10 are substantially less polar, despite the fact that the composition of these coatings is otherwise identical with those of Examples 1, 2 and 4.

Furthermore, data disclosed in “Evaluation of a poly(vinylpyrollidone)-coated biomaterial for urological use”; M. M. Tanney, S. P. Gorman, Biomaterials 23 (2002), 4601-4608, show that the contact angle of polyurethane is about 97° and that of PVP-coated polyurethane is about 50°.

### Example 12

### Measurement of Coagulation Parameters

A film for blood contact studies was produced by spin-coating the polyurethane dispersion of Example 1 onto glass. The sample surface was inserted into an autoclaved incubation chamber and incubated with 1.95 ml of blood. The exact experimental set-up is described in U. Streller et al. J. Biomed. Mater. Res B, 2003, 66B, 379-390.

The venous blood required for the test was withdrawn via a 19 G cannula from a male donor who had not taken any medicaments for at least 10 days. Coagulation was prevented by the addition of heparin (2 IU/ml). The thus-prepared blood was then inserted into the incubation chamber equipped with the polyurethane surface and preheated to 37° C., and was incubated for 2 h with permanent rotation of the chamber at 37° C. Comparison materials used were glass and polytetrafluoroethylene (PTFE). Glass is a strongly activating surface for blood coagulation, while PTFE is a polymer which for many applications is an acceptable material (see U. Streller et al. J. Biomed. Mater. Res B, 2003, 66B, 379-390).

After incubation had taken place, three parameters were measured:

Thrombin-antithrombin complex (Enzygnost TAT micro, Dade Behring GmbH, Marburg, Germany)

Platelet factor 4 (ELISA PF 4 complete kit from Haemochrom Diagnostica GmbH, Essen, Germany)

The thrombocyte reduction was measured in blood containing EDTA anticoagulant by means of an automatic cell counting system (AcTdiff from Coulter, Krefeld, Germany).

TABLE 2
Thrombin-antithrombin complex

| Surface | Thrombin-antithrombin complex (μg/mL) |
|---|---|
| Polyurethane of Example 1 | 27.7 |
| PTFE | 33.4 |

TABLE 3
Platelet factor 4

| Surface | Thrombin-antithrombin complex (IU/mL) |
|---|---|
| Polyurethane of Example 1 | 29.7 |
| Glass | 377.2 |
| PTFE | 59.2 |

TABLE 4
Thrombocyte reduction in the blood

| | Thrombocyte count |
|---|---|
| Surface | (% reduction) |
| Polyurethane of Example 1 | −0.3 |
| Glass | 17.9 |
| PTFE | 5.7 |

All three blood parameters measured show that the hydrophilic polyurethane of Example 1 activates coagulation only to a very moderate extent. The thrombin-antithrombin complex, as a measure of the activation of the intrinsic coagulation cascade, shows that the polyurethane, even in comparison to PTFE, which is regarded as being very highly blood-compatible, produces lower values and, as a result, induces an even lower activation.

Platelet factor 4 is a marker for the activation of the thrombocytes. This cellular part of the coagulation as well is activated only to a small extent by the hydrophilic polyurethane. The highly blood-compatible PTFE induces a higher activation. The reduction in thrombocytes as well is significant for glass and PTFE as well, which means that some of the thromobocytes attach to these surfaces. In the case of the hydrophilic polyurethane of Example 1, in contrast, there is virtually no reduction apparent.

### Example 13

This example describes the synthesis of an aqueous dispersion with terminal polyethylene oxide units as a comparison material to the inventive examples using a polyurethane terminated by a copolymer comprising polyethylene oxide and polypropylene oxide. The Polyether LB 25 used for the purposes of the present invention is replaced in this example by equal molar amounts of a comparable pure polyethylene oxide ether.

277.2 g of Desmophen C 2200 29.4 g of Polyethylene Glycol 2000 monomethyl ether (source: Fluka, CAS No. 9004-74-4) and 6.7 g of neopentyl glycol were introduced at 65° C. and homogenized by stirring for 5 minutes. At 65° C., this mixture was admixed over the course of 1 minute first with 71.3 g of 4,4′-bis(isocyanatocyclohexyl)methane (H₁₂MDI) and then with 11.9 g of isophorone diisocyanate. This mixture was heated to 110° C. After 35 min the theoretical NCO value was reached. The completed prepolymer was dissolved at 50° C. in 711 g of acetone and then at 40° C. a solution of 4.8 g of ethylene diamine in 16 g of water was metered in over the course of 10 min. The subsequent stirring time was 5 min. Subsequently, over the course of 15 min, a dispersion was carried out by addition of 590 g of water. After that the solvent was removed by distillation under reduced pressure. This gave a storage-stable polyurethane dispersion having a solids content of 40.0% and an average particle size of 130 nm.

As described under Example 11, a coating on glass was produced by spincoating, and the static contact angle of this coating was ascertained. The result obtained was a static contact angle of 45°. Comparing this figure with the figure for the coating of Example 1 (<10°, see Table 1 in Example 11) shows that the use of the mixed polyethylene oxide polypropylene oxide Monol LB 25 in comparison to the pure polyethylene oxide monol allows significantly lower contact angles and hence more hydrophilic coatings.

### Example 14

This example describes the synthesis of the polyurethaneurea polymer of Inventive Example 1 as a comparative example in organic solution.

A mixture of 277.2 g of Desmophen C 2200, 33.1 g of LB 25, 6.7 g of neopentyl glycol is admixed at 60° C. with 71.3 g of 4,4′-bis(isocyanatocyclohexyl)methane (H₁₂MDI) and 11.9 g of isophorone diisocyanate. The mixture was heated to 110° C. and reacted until a constant NCO content of 2.4 was obtained. The mixture was cooled and diluted with 475 g of toluene and 320 g of isopropanol. At room temperature, a solution of 4.8 g of ethylene diamine in 150 g of 1-methoxypropan-2-ol was added over the course. Following complete addition, stirring was continued for 2 h. This gave 1350 g of a 30.2% strength polyurethaneurea solution in toluene/isopropanol/1-methoxypropan-2-ol, having a viscosity of 607 mPas at 23° C.

As described under Example 11, a coating on glass was produced by spincoating, and the static contact angle of this coating was ascertained. The result obtained was a static contact angle of 27°. Comparing this figure with the figure for the coating of Example 1 (<10°, see Table 1 in Example 11), a structurally identical coating but in dispersion in water, shows that the coatings from aqueous dispersion, in comparison to coatings obtained starting from corresponding solutions, produce more hydrophilic coatings.

1.-10. (canceled)

11. A medical device having at least one coating comprising at least one polyurethaneurea wherein the polyurethaneurea is terminated with a copolymer unit comprising polyethylene oxide and polypropylene oxide.

12. The medical device according to claim 11, wherein the polyurethaneurea comprises units which originate from at least one hydroxyl-containing polycarbonate.

13. The medical device according to claim 11, wherein the polyurethaneurea comprises units which originate from at least one aliphatic, cycloaliphatic or aromatic isocyanate.

14. The medical device according to claim 11, wherein the polyurethaneurea comprises units which originate from at least one diamine or amine alcohol.

15. The medical device according to claim 11, wherein the polyurethaneurea is synthesised from components comprising
a) at least one polycarbonate polyol having an average molar weight between 400 g/mol and 6000 g/mol and a hydroxyl functionality of 1.7 to 2.3, or mixtures of such polycarbonate polyols;
b) at least one aliphatic, cycloaliphatic or aromatic polyisocyanate or mixtures of such polyisocyanates in an amount per mole of the polycarbonate polyol of 1.0 to 4.0 mol;
c) at least one monofunctional mixed polyoxyalkylene ether comprising polyethylene oxide and polypropylene oxide or a mixture of such polyethers, having an average molar weight between 500 g/mol and 5000 g/mol, in an amount per mole of the polycarbonate polyol of 0.01 to 0.5 mol;
d) at least one aliphatic or cycloaliphatic diamine or at least one amino alcohol, as so-called chain extenders, or mixtures of such compounds in an amount per mole of the polycarbonate polyol of 0.05 to 3.0 mol;
e) optionally, one or more short-chain aliphatic polyols having a molar weight between 62 g/mol and 500 g/mol, in an amount per mole of the polycarbonate polyol of 0.1 to 1.0 mol; and
f) optionally, amine- or OH-containing units which are located on, and cap, the polymer chain ends.

16. A process for coating a medical device comprising coating the medical device with at least one polyurethaneurea wherein the polyurethaneurea is terminated with a copolymer unit comprising polyethylene oxide and polypropylene oxide.

17. The process according to claim 16, wherein the coating is applied to the medical device by knifecoating, printing, transfer coating, spraying, spin coating or dipping.

18. A medical device obtained according to the process of claim 16.

19. The medical device according to claim 18, wherein the medical device is a contact lens; a cannula; a catheter; an embolism filter; a vena caval filter; endoscope; laryngoscope; tracheal device; a guide rod; an insertion guide; a vascular plug; a pacemaker component; a drainage tube; a guide wire; a glove; a stent; a membrane; a blood filter; a device for circulatory support; a dressing material for wound management; a urine bag; a stoma bag; or a feeding tube.

20. The medical device according to claim 19, wherein the medical device is an implant comprising a medically active agent, an implant an extracorporeal blood line, a cochlear implant, or a dental implant tube for feeding.

21. The medical device according to claim 19, wherein the medical device is a urological catheter; a central venous catheter; an inlet catheter; an outlet catheter; a catheter for angioplasty; a catheter for biopsy; a catheter used for introducing a stent; a bronchoalveolar lavage catheter; a balloon catheter; or a catheter used in coronary angioplasty.

22. The medical device according to claim 20, wherein the medical device is a urinary catheter or a ureteral catheter.

23. The medical device according to claim 19, wherein the medical device is a dilation balloon.

24. The medical device according to claim 19, wherein the medical device is an endotracheal tube, a respirator or a tracheal aspiration device.

25. The medical device according to claim 19, wherein the medical device is a membrane for dialysis.

26. The medical device according to claim 19, wherein the medical device comprises a medically active agent for a stent, for a balloon surface, or for a contraceptive.

27. A medical device according to claim 19 in the form of a radioactive stent, a drug-coated stent, bioabsorbable stent or a healing stent.
